(19) United States
(12) Patent Application Publication
(10) Pub. No.: US 2006/0099680 A1
(43) Pub. Date: May 11, 2006

(54) Yeast transformant into which genes associated with synthesis system of O-fucosylated protein are introduced

(75) Inventors: Yoshifumi Jigami, Tsukuba-shi (JP); Yuuko Chigira, Tsukuba-shi (JP); Xiao-Dong Gao, Tsukuba-shi (JP); Si jun Dong, Tsukuba-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Nat Inst of Adv Industrial Sci and Tech, Tokyo (JP)

(21) Appl. No.: 11/113,081

(22) Filed: Apr. 25, 2005

(30) Foreign Application Priority Data
Apr. 23, 2004 (JP) 2004-128796
Apr. 15, 2005 (JP) 2005-118146

(51) Int. Cl. C12P 21/06; C07H 21/04; G01N 33/00; C12N 15/74; C12N 1/18; C07K 14/39

(52) U.S. Cl. 435/69.1; 435/254.2; 435/483; 536/23.2; 530/395; 436/86

(57) ABSTRACT

An object of the present invention is to provide a means for producing O-fucosylated protein in large quantities, a means for searching for new genes associated with the synthesis system of O-fucosylated protein or the proteins expressed by the genes, and a means for elucidating the functions thereof. According to the present invention, a yeast transformant characterized in that the genes associated with the synthesis system of O-fucosylated protein are a GDP-fucose synthase gene, a GDP-fucose transporter gene, a fucosyltransferase gene, and a fucose receptor gene is provided.

### FIELD OF THE INVENTION

The present invention relates to a yeast transformant into which genes associated with the synthesis system of O-fucosylated protein are introduced, a method for producing O-fucosylated protein using the yeast transformant, a yeast transformant wherein some of the genes associated with the synthesis system are deleted, a screening method for genes associated with the synthesis system of O-fucosylated protein using the yeast transformant, a gene kit and recombinant plasmids for preparing the yeast transformant.

### BACKGROUND OF THE INVENTION

Glycostructure linked to protein has been revealed to be very important for the functions relating to the biological activity of protein. Recently, numerous therapeutic agents have been actively developed based on protein by proteomics or genomics. For the expression of the functions of such therapeutic proteins, sugar chain modification that is one of posttranslational modifications, is essential in most cases. As mammalian sugar chain modification, Asn-linked-type, mucin-type, or proteoglycan-type modification, or the like, are mainly well known (see Makoto Takeuchi, Glycobiology Series 5, Glycotechnology, edited by Yo Kohata, Senichiro Hakomori, and Katsutaka Nagai, Kodansha Scientific, 191-208 (1994)). Each modification type forms a unique glycostructure through a different biosynthesis pathway.

Most therapeutic proteins are currently expressed and produced by animal cells. However, many inconveniences can be noted with respect to expression by animal cells, when nonuniformity of production amounts or products, culture cost, contamination with viruses, times required for stable obtainment of cells for production, and the like are taken into consideration. Despite such inconveniences, expression and production by animal cells still offer significant advantages. This is because protein expressed in animal cells is subjected to various natural mammalian complicated sugar chain modifications, is rapidly incorporated into the body as a therapeutic agent, and then exerts its functions.

To overcome the above problems in animal cells, some therapeutic proteins are produced at extremely low cost by expression systems of prokaryotes such as Escherichia coli. However, prokaryotes generally lack metabolic pathway for protein modification with sugar chains. That is, a host for enabling sugar chain modification should be at least eukaryotes. Hence, protein expression systems using alternative hosts such as plant cells, insect cells, and yeast cells are currently being examined. Of these, yeast is easy to handle, because it is a unicellular organism as well as is a eukaryote. Thus yeast has been widely utilized for a long time as a model organism in the field of research. The largest number of findings have been accumulated concerning yeast metabolic pathways. Alteration of yeast for use as alternative hosts after elucidation of the basic glycostructure means that such alternation leads to development of therapeutic agents at a low cost.

Asn-linked sugar chain is synthesized by the same mechanism up to the stage of the endoplasmic reticulum in both mammals and yeasts. In the endoplasmic reticulum, glycoprotein is synthesized, to which M8 high mannose-type sugar chain (Man8GlcNAc2) consisting of mannose 8 residue and N-acetylglucosamine 2 residue is linked. Modifications in the subsequent stage of the Golgi apparatus and the following stages originally differ completely between mammals and yeast. Recently, Asn-linked sugar chain has been altered to that of the human type through yeast gene engineering techniques. This is an attempt to utilize yeast cells as hosts for producing glycoprotein therapeutic agents, whereby alteration at the gene level is easier than with the use of animal cells and low-cost culture and improved productivity can be expected.

In the Golgi apparatus of mammalian cells, the above Man8GlcNAc2 structure synthesized in the endoplasmic reticulum is not altered, is altered to glycoprotein having acidic sugar chains as a result of addition of phosphate groups, or is modified with various sugar units referred to as hybrid-type and complex-type. However, sugar chain modification in yeast Golgi apparatus results in the generation of outer sugar chains wherein several to 100 or more residues of mannose are added to Man8GlcNAc2. It is known that when an OCH1 gene encoding an enzyme responsible for the initial mannosyltransfer reaction is disrupted so as to delete the very large mannose structure, glycoproteins wherein only the Man8GlcNAc2 structure has been added are produced also in yeast (see Nakayama et al., EMBO J., 11, 2511-2519 (1992)). Furthermore, research has also been conducted using an OCH1-disrupted strain for developing sugar chains that are more analogous to the hybrid-type or complex-type sugar chains in mammalian cells. Examples of such research include a case where trimming of the Man8GlcNAc2 structure has been attempted by introducing foreign mannosidase, thereby realizing a core structure that is closer to that of a mammalian human type (see Chiba et al., J. Biol. Chem., 273, 26298-26304 (1998)) and a case where GlcNac has further been transferred to the trimmed core structure utilizing yeast endogenous nucleotide sugar (see Choi et al., Proc. Natl. Acad. Sci. U.S.A. 5022-5027 (2003)).

Complex-type sugar chains in mammalian cells have fucose and sialic acid as sugar units with very important functions. In particular, the transfer of fucose increases the number of branches of complex-type sugar chains, creating highly variable branching structures of such sugar chains. Moreover, in the case of mucin-type sugar chains, carcinoma-related carbohydrate antigens such as sialyl Lewis A and sialyl Lewis X antigens are known as fucose-containing structures. The importance of fucose in glycoprotein has been revealed.

However, sugar units such as fucose and sialic acid are absent in glycoprotein derived from yeast. This is because yeast genes do not encode any enzyme group associated with the metabolic system for transfering these sugars to protein. To develop production of useful glycoprotein using yeast as a host, it is required to enable formation of a glycostructure containing these sugar units having important functions in mammalian cells. With existing technology only, it is impossible to create a glycostructure comprising fucose and sialic acid in yeast. To make it possible to create such a glycostructure in yeast, it is required first to create, within yeast cells, a pool of nucleotide sugar as starter material, such as GDP-fucose or CMP-sialic acid that are sugar donors for glycosyltransferase that are absent in yeast. This has been a heavy drag on the creation of such a glycostructure. Regarding this technology, we have recently transplanted the de novo synthesis pathway from GDP-mannose into yeast, which is well conserved in organisms ranging from mammals to plants, thereby succeeding in accumulation of GDP-fucose within yeast cells (see JP Patent Publication (Kokai) 2001-145488 A). Realization of effective utilization of GDP-fucose makes it possible to create a fucose-linked glycostructure in yeast. However, with conventional technology, it has been impossible to utilize in vivo GDP-fucose that is originally absent in yeast.

Meanwhile, it has been revealed that mammalian cells have a glycostructure wherein fucose is directly linked via oxygen to serine/threonine residues in protein. Such an O-linked fucose structure has been discovered from a membrane protein that is mainly present in/on cell surface layers. It has also been revealed that the structure is involved in cell-to-cell interaction and signal transduction. Many O-linked fucoses are present in the extracellular domains of Notch receptors that regulate Notch signals. Such a glycostructure containing fucoses is essential in the Notch signal transduction. It has become clear that the Notch signal transduction is controlled in complicated ways depending on the structure or proportion of sugar chains (see Okajima et al., Cell 111, 893-904 (2002)). Furthermore, it has been revealed that extracellular domains of Notch ligands have a similar structure (see Panin et al., J. Biol. Chem., 277, 29945-29952 (2002)). Recently, the role of O-linked fucose is increasingly interesting, as is that of fucoses contained in N-linked sugar chains. Also for the purpose of elucidating the relationship between glycostructures and functions including the physiological activity of glycoproteins, if the above-mentioned glycoproteins having such a rare structure can be obtained in large quantities, new findings can be clearly obtained.

Moreover, expression systems using yeast as hosts have been used for identifying the functions or properties of proteins. Genes of many foreign proteins are introduced into yeast by such systems. Of these cases, one of the conventional methods for measuring the activity of nucleotide sugar transporters for transporting nucleotide sugar in animals including humans or in plants is carried out by obtaining the nucleotide sugar transporters as recombinant proteins in yeast, and then indirectly measuring the activity of transporting nucleotide sugar as substrates by the utilization of yeast's Golgi apparatus fraction that is the glycoprotein synthesis system of yeast. However, some of nucleotide sugars cannot be metabolized in the yeast glycoprotein synthesis depending on their type. When such nucleotide sugar is used as a substrate with which the yeast glycoprotein synthesis system does not function, transport activity analyzed in this case will be at a very low level. For example, within natural animal or plant cells, their own nucleotide sugar transporters express their function to actually transport nucleotide sugar used as a test substrate. However, such a measurement method using the yeast's Golgi apparatus fraction involves a risk that the transport activity determined in this case will be at a low level. Hence, precise identification of a substrate showing the highest transport activity requires much time, labor, and cost.

### SUMMARY OF THE INVENTION

In view of the current circumstances of the above prior art, an object of the present invention is to provide a means for producing O-fucosylated protein in large quantities in yeast and a new means for searching for genes associated with the synthesis system of O-fucosylated protein or the proteins expressed by the genes.

As a result of intensive studies to achieve the above object, we have further advanced our prior developmental product, the technology for producing GDP-fucose within yeast cells. Specifically, we have newly introduced genes associated with the synthesis system of O-fucosylated protein into yeast, which are originally absent in yeast, thereby succeeding in efficient production of O-fucosylated protein in yeast. Furthermore, we have also discovered that the newly constructed synthesis system of O-fucosylated protein in yeast can be effectively utilized as a new means for searching for useful genes associated with the synthesis system of O-fucosylated protein or the proteins expressed by the genes, thereby completing the present invention.

The present invention relates to the following (1) to (20).

(1) A yeast transformant, wherein genes associated with a synthesis system of O-fucosylated protein are introduced.

(2) The yeast transformant of (1), wherein the genes associated with the synthesis system of O-fucosylated protein are a GDP-fucose synthase gene, a GDP-fucose transporter gene, a fucosyltransferase gene, and a fucose receptor gene.

(3) The yeast transformant of (2), wherein the fucose receptor gene has a secretion signal sequence added thereto.

(4) The yeast transformant of (3), wherein the fucose receptor gene is a DNA containing a nucleotide sequence encoding at least an EGF domain.

(5) A recombinant vector, wherein a GDP-fucose transporter gene is inserted under control of a promoter for yeast.

(6) A recombinant vector, wherein a fucosyltransferase gene is inserted under control of a promoter for yeast.

(7) A recombinant vector, wherein a fucose receptor gene is inserted under control of a promoter for yeast.

(8) The recombinant vector of (7), wherein the fucose receptor gene has a secretion signal sequence added thereto.

(9) The recombinant vector of (8), wherein the fucose receptor gene is a DNA containing a nucleotide sequence encoding at least an EGF domain.

(10) A recombinant vector, wherein a GDP-fucose synthase gene, a GDP-fucose transporter gene and/or a fucosyltransferase gene are inserted under control of a promoter for yeast.

(11) A use of the recombinant vector of any one of (5) to (10) for preparing the yeast transformant of any one of (1) to (4).

(12) A method for producing O-fucosylated protein, which comprises culturing the yeast transformant of any one of (1) to (4) and collecting O-fucosylated protein from the culture product.

(13) A gene kit for preparing the yeast transformant of any one of (1) to (4), which contains a GDP-fucose synthase gene, a GDP-fucose transporter gene, a fucosyltransferase gene, and a gene encoding a fucose receptor.

(14) A yeast transformant, wherein the following genes (a) or (b) are introduced:

(a) a GDP-fucose synthase gene and a GDP-fucose transporter gene; or

(b) a GDP-fucose synthase gene, a fucosyltransferase gene, and/or a fucose receptor gene.

(15) A method for screening for genes associated with the synthesis system of O-fucosylated protein and/or the proteins expressed the genes, which comprises culturing the yeast transformant of (14), collecting the expressed proteins, and screening for the genes and/or the proteins using the presence of sugar molecules in the expressed proteins as an indicator.

(16) A method for confirming or measuring the activity of proteins expressed by genes associated with the synthesis system of O-fucosylated protein, which comprises culturing the yeast transformant of (14), collecting the expressed proteins, and confirming or measuring the activity using the presence of sugar molecules in the expressed proteins as an indicator.

(17) A gene kit for preparing the yeast transformant of (14), which contains the following genes (a) or (b):

(a) a GDP-fucose synthase gene and a GDP-fucose transporter gene; or

(b) a GDP-fucose synthase gene, a fucosyltransferase gene, and/or a fucose receptor gene.

(18) A method for measuring the number of disulfide bonds in protein, which comprises culturing the yeast transformant of any one of (1) to (4), collecting O-fucosylated protein from the culture product, and measuring the molecular weights of the obtained O-fucosylated protein and protein with no fucose linked thereto to find an increase in molecular weight as a result of the linkage of fucose to protein.

(19) A protein, which has the amino acid sequence represented by SEQ ID NO: 8 in the sequence listing or an amino acid sequence derived from such amino acid sequence by deletion, substitution, or addition of 1 or several amino acids, and which has GDP-fucose transporter activity.

(20) A gene, which encodes the protein of (19).

### BRIEF DESCRIPTION OF DRAWINGS

FIG. 1 is a schematic diagram showing the synthesis process of O-fucosylated protein in yeast.

FIG. 2 is a schematic diagram showing the structures of recombinant vectors for expressing each gene associated with the synthesis system of O-fucosylated protein.

FIG. 3 shows the nucleotide sequences of synthetic DNAs used for constructing EGF domains.

FIG. 4 shows photographs showing the results of confirming the expression of each gene associated with the synthesis system of O-fucosylated protein in yeast (S. cerevisiae) by Western blotting [M: molecular weight marker; lane 1: wild strain (W303-1A); lane 2: transformed strain (YCH10); and lane 3: transformed strain (YCH09)].

FIG. 5 shows photographs showing the results of confirming the expression of each gene associated with the synthesis system of O-fucosylated protein in yeast (P. pastoris) by Western blotting [wild strain (SM1168) and transformed strain (SDP05)].

FIG. 6 shows photographs showing the results of confirming the presence of EGF domains in specimens purified from the culture products of an S. cerevisiae yeast transformed strain by SDS-PAGE, CBB staining (A), and then Western blotting (B) using anti-His antibodies [lane 1: control strain (YCH04); lane 2: transformed strain (YCH10); and lane 3: transformed strain (YCH09)].

FIG. 7(A) is a photograph showing the results of confirming the linkage of fucose to EGF domain specimens purified from the culture product of an S. cerevisiae yeast transformed strain by lectin blotting using a fucose-recognizing lectin [lane 1: control strain (YCH04); lane 2: transformed strain (YCH10); and lane 3: transformed strain (YCH09)]. FIG. 7(B) is a chart diagram showing the results of HPLC conducted on EGF domain specimens purified from the culture products of a control strain (YCH04) and a transformed strain (YCH10). FIG. 7(C) shows the results of molecular weight analysis conducted with MALDI-TOF-MS on EGF domain specimens sampled from each peak (P1 and P2) of HPLC.

FIG. 8 shows photographs showing the results of confirming the presence of EGF domains in specimens purified from the culture product of a P. pastoris yeast transformed strain by SDS-PAGE, CBB staining (A), and then Western blotting (B) using anti-His antibodies [transformed strain (SDP05) and wild strain (SM1168)].

FIG. 9 is a chart diagram showing the results of HPLC conducted on EGF domain specimens purified from the culture product of a P. pastoris yeast transformed strain [specimen (A) purified from the culture product of a control strain (SDP03) and specimen (B) purified from the culture product of the transformant (SDP05) caused to express all the genes].

The present invention is explained in detail as follows. This application claims priority of Japanese patent application No. 2004-128796 filed on Apr. 23, 2004, and that of Japanese patent application No. 2005-118146 filed on Apr. 15, 2005, and it encompasses the content described in the specifications of these patent applications.

As described above, we have already developed technology for causing the production of GDP-linked fucose (GDP-fucose) within yeast cells. However, yeast lacks the synthesis system of glycoprotein using the nucleotide sugar as a substrate.

According to the present invention, through introduction into yeast of a plurality of genes associated with the synthesis system of O-fucosylated protein using the GDP-fucose as a substrate, a yeast transformant having the protein synthesis system constructed therein is obtained in yeast and O-fucosylated protein is produced using the yeast transformant.

1. Genes Associated with the Synthesis System of O-Fucosylated Protein

According to the present invention, a yeast transformant that can produce O-fucosylated protein is provided. The yeast transformant can be prepared by introducing genes associated with the synthesis system of O-fucosylated protein into yeast. Examples of genes associated with the synthesis system of O-fucosylated protein include a GDP-fucose synthase gene, a GDP-fucose transporter gene, a fucosyltransferase gene, and a fucose receptor gene.

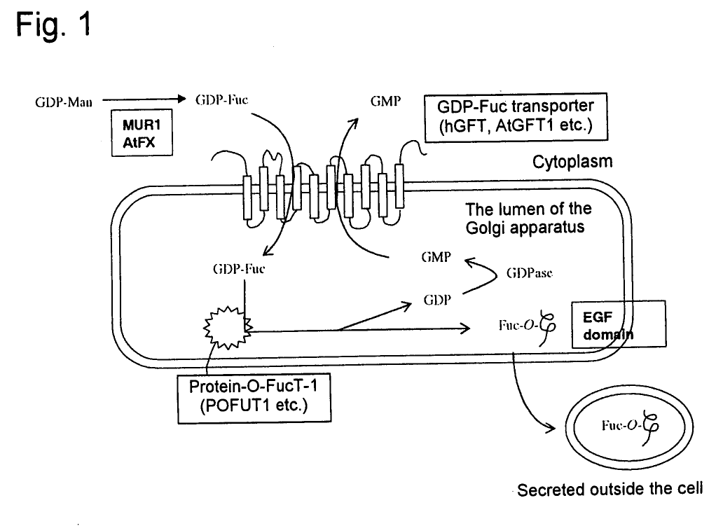

The present invention will be hereafter explained by referring to FIG. 1.

FIG. 1 shows the synthesis process of O-fucosylated protein in yeast.

To cause the production of O-fucosylated protein in yeast, in the present invention, first, GDP-fucose to be used as a substrate is accumulated within the yeast cytoplasm by the above means that we have developed. For this purpose, a GDP-fucose synthase gene is introduced into yeast. As the GDP-fucose synthase gene, for example, 2 types of enzyme gene, Arabidopsis thaliana MUR1 gene and AtFX gene, can be used. The intensity of the 2 types of enzyme expressed by these genes makes it possible to impart ability to convert GDP linked mannose (GDP-mannose) generated within yeast cells to GDP-fucose. The nucleotide sequence and the corresponding amino acid sequence of MUR1 gene and the same of AtFX gene are shown in SEQ ID NOS: 1, 2, 3, and 4, respectively, in the sequence listing.

Next, to impart ability to yeast to transport GDP-fucose accumulated within the yeast cytoplasm into the Golgi apparatus (where glycosyltransfer reaction and glycoprotein synthesis take place), a GDP-fucose transporter gene is introduced into yeast. Thus, GDP-fucose is transported to and incorporated in the lumen of the Golgi apparatus in yeast. As such a GDP-fucose transporter gene, for example, a human-derived gene (hGFT) or an Arabidopsis thaliana-derived gene (AtGFT1) can be used.

The above Arabidopsis thaliana-derived AtGFT1 is a gene that was newly isolated after a search of the cDNA library of Arabidopsis thaliana for the purpose of expressing functions at high efficiencies in yeast. Search for the transporter gene can be carried out by picking up 8 proteins having homology with a known GDP-fucose transporter from the database of Arabidopsis thaliana, producing a comprehensive phylogenetic tree including these proteins in addition to other nucleotide sugar transporters, and, in addition, referring to a hydrophobic plot or the like wherein orientation with the membrane is taken into consideration.

The nucleotide sequence and the corresponding amino acid sequence of the above hGFT gene and the same of the novel AtGFT1 gene isolated from Arabidopsis thaliana are shown in SEQ ID NOS: 5, 6, 7, and 8, respectively, in the sequence listing.

Furthermore, in the present invention, to utilize GDP-fucose as a sugar donor, a fucosyltransferase gene and a gene expressing the receptor thereof are introduced in combination into yeast. Currently 8 fucosyl transferases are known as synthases of Asn-linked and mucin-type sugar chains in mammals. However, yeast lacks such glycosyltransferases and endogenous substrates that can serve as sugar receptors thereof. Hence, in the present invention, first a fucose receptor that can be expressed in yeast is examined. As the fucose receptor, it was considered that the structure of an EGF (epidermal growth factor) domain contained in a Notch receptor or Notch ligand is preferable. Examples of protein containing the O-fucose structure in an EGF domain include urokinase and a human coagulation factor. As receptors, their EGF domains can be utilized or protein containing the structure can be directly utilized. Although no enzyme responsible for a transfer reaction has yet been identified, the O-fucose structure is also present in the TSR module in thrombospondin. Combinations of these unknown transferases and receptors can be appropriately utilized. By causing co-expression of such a receptor and glycosyltransferase in yeast, a fucosyltransfer reaction system can be newly constructed within yeast. Thus, it becomes possible to produce O-fucosylated protein by carrying out a fucosyltransfer reaction in yeast using GDP-fucose as a substrate.

Protein-O-Fucosyltransferase-1 (POFUT1) has been isolated as a gene encoding an enzyme responsible for a fucosyltransfer reaction into EGF domains. In the O-linked fucose structure, fucose is linked to specific serine and/or threonine that is present in conserved sequences in EGF domains. An EGF domain contains 6 cysteines that form 3 specific disulfide bonds. These 3 disulfide bonds are essential for the expression of the activity of POFUT1 that has been reported as an enzyme that transfers fucose. In synthetic peptides consisting of conserved sequences, no fucosylation takes place. Hence, in terms of 1) requiring the entire domain and 2) requiring specific disulfide bonds in the domain, significant efforts are required for the expression of the activity of POFUT1 using a receptor synthesized by organic chemical methods. In the present invention, EGF domains are synthesized within yeast cells. Thus, it becomes possible to readily prepare EGF domains having an appropriate folding structure as fucose receptors only through causing co-expression with POFUT1. The nucleotide sequence and the corresponding amino acid sequence of the POFUT1 gene are shown in SEQ ID NOS: 9 and 10 in the sequence listing.

In the present invention, when an EGF domain is prepared as a fucose receptor, a gene is designed so as to contain a nucleotide sequence at least encoding the EGF domain, and then the gene is introduced into yeast. The nucleotide sequence and the corresponding amino acid sequence of an EGF domain are shown following histidine tags in the sequences of SEQ ID NOS: 11 and 12 in the sequence listing.

In addition, combinations of a fucosyltransferase gene and a fucose receptor gene to be introduced into yeast are not limited to the above, as long as their activity is expressed in yeast.

Moreover, in the present invention, a secretion signal sequence derived from yeast is preferably added to a fucose receptor gene. Accordingly, for example, an O-fucose-linked EGF domain is generated by fucosyltransferase in the lumen of the Golgi apparatus, and transported into a medium from the endoplasmic reticulum lumen and the lumen of the Golgi apparatus via a secretory pathway. As a result, O-fucosylated protein can be produced in a highly efficient manner. As a secretion signal derived from yeast, conventionally known signals can be used. An example of such a signal is an α-factor precursor of budding yeast. A fusion product of a prepro sequence of a yeast α-factor and O-fucosylated protein is thus expressed. The prepro sequence in the fusion product is removed when the fusion product is secreted outside the yeast, so that O-fucosylated protein is obtained.

A gene sequence and the corresponding amino acid sequence encoding an EGF domain having an α-factor and a histidine tag added thereto are shown in SEQ ID NOS: 11 and 12 in the sequence listing.

The amino acid sequence of the protein encoded by each of the above genes associated with the synthesis system of O-fucosylated protein may be an amino acid sequence derived from the amino acid sequence (SEQ ID NO: 2, 4, 6, 8, 10, or 12) by deletion, substitution, or addition of 1 to several amino acids, as long as it retains functions equivalent to those of the original protein. Here, “1 to several” ranges from, but is not specifically limited to, for example, approximately 1 to 20, preferably 1 to 10, more preferably 1 to 7, further preferably 1 to 5, and particularly preferably 1 to 3 amino acids.

Furthermore, examples of the protein encoded by each of the above genes associated with the synthesis system of O-fucosylated protein also include a protein that is functionally equivalent to the protein and has homology with the amino acid sequence of the protein. “Protein having homology” means a protein having homology of approximately 70% or more, preferably approximately 80% or more, more preferably approximately 90% or more, and most preferably approximately 95% or more with the amino acid sequence of SEQ ID NO: 2, 4, 6, 8, 10, or 12. Protein homology can be determined according to the algorithm described in the document (Wilbur, W. J. and Lipman, D. J., Proc. Natl. Acad., Sci. U.S.A. (1983) 80, 726-730).

Each of the above genes associated with the synthesis system of O-fucosylated protein is amplified by the PCR method using, for example, a genome DNA gene or a cDNA library containing the gene region as a template, sense and antisense primers designed to obtain target gene fragments, and thermostable polymerase. Thus, each gene can be amplified by hundreds of thousands-fold or more within approximately 2 to 3 hours. As primers, 25 to 30 mer synthetic single-stranded DNAs are used. However, synthesis may be carried out not only by the PCR method but also by other methods.

Each of the above genes associated with the synthesis system of O-fucosylated protein can be previously combined with other materials for preparing yeast transformants so as to prepare a kit. For example, such a kit can include primers for amplifying the gene, vectors for introducing the gene, yeast cells, media or containers for culturing the cells, instructions describing the method for using such a kit, and the like, in addition to the above gene.

2. Recombinant Vector

The present invention involves introduction of a combination of the above genes associated with the synthesis system of O-fucosylated protein into yeast so as to obtain yeast transformants and to form the synthesis system of O-fucosylated protein in yeast. For introduction of these genes, for example, a gene cassette is prepared by ligating a gene ORF prepared by the PCR method or the like downstream of a promoter (hereinafter, referred to as a yeast promoter) that can be expressed in yeast, thereby preparing a recombinant vector for expression in yeast.

Examples of a recombinant vector of the present invention include: a recombinant vector wherein either a GDP-fucose transporter gene or a gene encoding fucose receptor protein is inserted under control of a yeast promoter; and a recombinant vector wherein a GDP-fucose synthase gene, a GDP-fucose transporter gene, and/or a fucosyltransferase gene are inserted under control of a yeast promoter.

Regarding preparation of these recombinant vectors, such recombinant vectors can be constructed by, for example, adding appropriate restriction enzyme sites to both ends of genes prepared by the above PCR method or the like and using proper restriction enzymes and DNA ligase. DNA once introduced into a plasmid can be readily amplified, isolated, and purified using Escherichia coli. Furthermore, determination or the like of the DNA sequences of genes can be carried out by a general method, such as a dideoxy method. Furthermore, determination of DNA sequences can also be conveniently carried out using a commercial sequencing kit or the like.

As yeast promoters, all the existing and known yeast promoters that are used for expression in yeast can be utilized. For example, gal1 promoters, gal10 promoters, PH05 promoters, PGK promoters, GAP promoters, ADH promoters, and AOX1 promoters are used.

Furthermore, a recombinant vector of the present invention may contain a replication initiation site (e.g., 2 μm DNA or a site derived from ARS1) and a selection marker (e.g., Leu2, Trp1, or Ura3) in addition to the above promoter. If necessary, the vector may further contain an enhancer, a terminator, a ribosome-binding site, a polyadenylation signal, and the like.

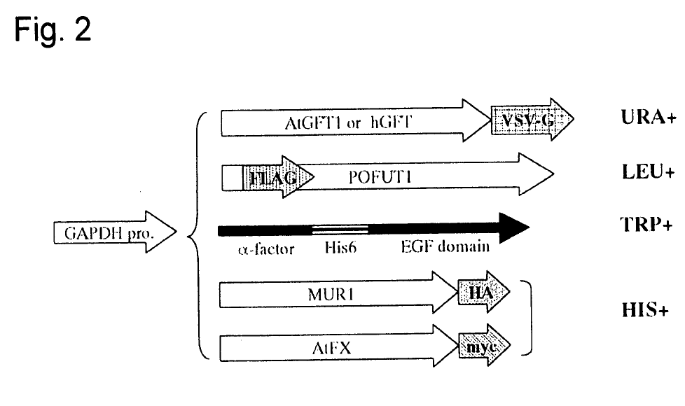

The structure of a recombinant vector in the present invention will be further described specifically by referring to FIG. 2.

Genes are designed so that they are expressed as proteins having different tags fused thereto to confirm the presence or the absence of protein expression. Examples of types of tag include, but are not specifically limited to, VSV-G, FLAG, histidine (His6), HA, and protein C. Regarding a tag to be fused to an EGF domain, tags with which protein can be readily purified are preferably selected. For example, the use of histidine (His6) enables affinity purification by the use of a carrier (nickel-chelated carrier) having affinity thereto.

At this time, regarding selection markers, plasmids are constructed utilizing different selection markers for all of the vectors, so that transformation by each vector can be confirmed in all cases. Moreover, when these foreign genes are incorporated onto yeast chromosomes, vectors having no autonomous replication sites are selected, plasmids are linearized by cleavage at restriction enzyme sites within marker genes, and then the resultants are introduced into yeast cells. Furthermore, as shown in the figures of MUR1 and AtFX, expression cassettes containing promoters can be incorporated into the same vector and then used.

3. Preparation of Yeast Transformant

Yeast transformants of the present invention can be obtained by introducing the above recombinant vectors into yeast. Examples of yeast used herein include Saccharomyces cerevisiae, Pichia pastoris, and Schizosaccharomyces pombe.

A method for introducing recombinant vectors into yeast is not specifically limited as long as it is generally used for introducing DNA into yeast. For example, a method that involves causing yeast hosts to incorporate recombinant vectors by homologous recombination, a lithium acetate method, an electroporation method, or spheroplast method may be employed.

The transformants obtained with such means are crossed based on tetrad analysis utilizing yeast genetics and then separated, so that yeast transformants wherein some or all of the above genes are introduced can be obtained.

4. Production of O-Fucosylated Protein

O-fucosylated protein, the object of the present invention, can be obtained by culturing yeast transformants wherein all of the above genes are introduced and then collecting the protein from the culture product thereof. “Culture product” means any of cultured cells or cultured microbial bodies or disrupted cells or disrupted microbial bodies in addition to culture supernatant.

A method for culturing yeast transformants of the present invention in media can be carried out according to a general method that is employed for culturing yeast. For example, as a medium for culturing yeast transformants, either a natural or a synthetic medium may be used, as long as it contains a carbon source assimilable by microorganisms, a nitrogen source, inorganic salts, and the like and enables efficient culture of transformants. A carbon source may be any such source that is assimilable by the organisms. For example, carbohydrates such as glucose, fructose, sucrose, and starch, organic acids such as acetic acid and propionic acid, and alcohols such as ethanol and propanol may be used. As a nitrogen source, in addition to ammonium salt of inorganic acid or organic acid, such as ammonia, ammonium chloride, ammonium sulfate, ammonium acetate, or ammonium phosphate, or other nitrogen-containing compounds, peptone, meat extracts, corn steep liquor, or the like may be used. As inorganic salts, primary potassium phosphate, secondary potassium phosphate, magnesium phosphate, magnesium sulfate, sodium chloride, ferrous sulfate, manganese sulfate, copper sulfate, calcium carbonate, or the like may be used.

After culture, when O-fucosylated protein of the present invention is produced within cells, the protein is extracted by disrupting the cells. Furthermore, when O-fucosylated protein of the present invention is produced outside the cells, the culture solution is directly used or the cells are removed by centrifugation or the like. Subsequently, through the use of one of or an appropriate combination of general biochemical methods that are used for isolation and purification of protein, such as ammonium sulfate precipitation, gel chromatography, ion exchange chromatography, and affinity chromatography, the protein of the present invention can be isolated and purified from the above culture product.

The presence or the absence of fucose in the obtained protein is confirmed utilizing blot analysis using fucose-recognizing lectin. To evaluate the presence or the absence of fucose, the sugar unit that is originally absent in yeast, any type of lectin for detection or any detection method may be used as long as positive results can be obtained, and they are not specifically limited.

5. Utilization of Yeast Transformant

The above yeast transformants wherein all or some of the genes associated with the synthesis system of O-fucosylated protein are introduced can be utilized for screening for novel genes associated with the synthesis system of O-fucosylated protein, measuring the number of disulfide bonds, screening for glycosyltransferases whose sugar receptors are protein substrates that are difficult to synthesize in vitro, confirming the activity of nucleotide sugar transporters whose transport substrates are difficult to specify, and the like.

For example, the above yeast transformants wherein some of the genes associated with the synthesis system of O-fucosylated protein are introduced can be used for screening for new genes having functions equivalent to those of genes that are not introduced. In the present invention, such a yeast transformant wherein some of the genes associated with the synthesis system of O-fucosylated protein are introduced means a yeast transformant wherein (a) a GDP-fucose synthase gene and a GDP-fucose transporter gene are introduced, or a yeast transformant wherein (b) a GDP-fucose synthase gene, a fucosyltransferase gene, and/or a fucose receptor gene are introduced. For example, the yeast transformant wherein the GDP-fucose synthase gene and the fucose transporter gene are introduced can be used for screening for a novel pair of a fucosyltransferase gene and a fucose receptor gene. The yeast transformant wherein the GDP-fucose synthase gene, the fucosyltransferase gene, and the fucose receptor gene are introduced can be used for screening for a novel GDP-fucose transporter gene.

Regarding screening, the above yeast transformants are cultured, the expressed proteins are collected and purified from the culture product, and then the presence of sugar molecules in the protein is confirmed. Thus, it is made possible to evaluate: 1) whether or not arbitrary nucleotide sugar has been transported to organelles where a transfer reaction takes place (activity of transporter); and 2) whether or not arbitrary glycosyltransfer reaction has taken place in the organelles where a transfer reaction takes place (activity of glycosyltransferase). Specifically, the transfer of fucose can be confirmed utilizing a separation method using chromatography or the like utilizing the fact that the linkage of fucose causes a change in polarity or a lectin column utilizing the linkage of fucose, or the like. Furthermore, the transfer of fucose can also be confirmed by measuring the molecular weight of purified receptor protein using MS and then directly measuring an increase in molecular weight as a result of the linkage of fucose.

### BEST MODES FOR CARRYING OUT THE INVENTION

Examples of the present invention will be given as follows. However, the present invention is not limited by these examples.

### EXAMPLE 1

### Preparation of Strain Wherein Each Gene Associated with the Synthesis System of O-Fucosylated Protein is Introduced

(1) Preparation of YCH01 Strain

An hGFT gene is located on human chromosome 20, and the cDNA nucleotide sequence of the hGFT gene has been registered under AF326199 in a database. The full-length cDNA of the hGFT gene was amplified by PCR using primer A (AATGAGCTCATGAATAGGGCCCCTCTGAAG: SEQ ID NO: 13) and primer B (ACTCTAGATCATTTACCCAATCTATTCATTTCAATATCAGTGTACACCCCCAT GGCGCTCTTC: SEQ ID NO: 14). A human brain-derived cDNA library (Clontech) was used as a template. Primer B was designed to encode a VSVG antigen (to be used for confirming expression), so that the thus amplified DNA fragment will be expressed as protein having the tag fused to the 3′ side. The PCR product was cleaved at the Sac I and Xba I site and then incorporated into the Sac I/Xba I site of a previously reported plasmid Yep352GAPII, thereby constructing a plasmid YEp352GAP-hGFT/VSVG whereby the hGFT gene was expressed in yeast. Next, a region containing a yeast GAPDH promoter and terminator was excised with BamH I from the plasmid, both ends were blunted, and then the resultant was inserted into the Pvu II site of a pRS306 integration vector. The plasmid pRS-hGFT/VSVG was cleaved with EcoR V and then the S. cerevisiae W303-1A strain was transformed. Transformation was carried out using a lithium acetate method. After transformation, the resultant was inoculated on an SD-Ura medium plate (2% glucose, 0.67% Yeast Nitrogen Base w/o amino acids (produced by Difco Laboratories), nucleic acid bases excluding uracil, and an amino acid mixture (20 to 400 mg/l)) and then cultured at 30° C. for 2 days, thereby obtaining transformants. The transformants were scraped off the plate and then subjected to a simple PCR method (whereby the transformants were suspended in a PCR reaction solution) so as to confirm incorporation onto the chromosome. The thus obtained transformants were determined to be of a YCH01 strain.

(2) Preparation of YCH02 Strain

An AtGFT1 gene is located on chromosome 5 of Arabidopsis thaliana. The cDNA nucleotide sequence of the AtGFT1 gene has been registered under At5g19980 in a database. The full-length cDNA of the AtGFT1 gene was amplified by PCR using primer C (AGAGCTCATGTCGTCCTCTCGATTCGAT: SEQ ID NO: 15) and primer D (CCTCTAGATCATTTACCCAATCTATTCATTTCAATATCAGTGTATACAACAG AAGCTAGTTTC: SEQ ID NO: 16). An Arabidopsis thaliana-derived cDNA library (Clontech) was used as a template. Primer D was designed to encode a VSVG antigen (to be used for confirming expression), so that the thus amplified DNA fragment will be expressed as protein having the tag fused to the 3′ side. The PCR product was cleaved at the Sac I and Xba I sites and then incorporated into the Sac I/Xba I site of a previously reported plasmid Yep352GAPII, thereby constructing a plasmid YEp352GAP-AtGFT1/VSVG whereby the AtGFT1 gene was expressed in yeast. Next, a region containing a yeast GAPDH promoter and terminator was excised with BamH I from the plasmid, both ends were blunted, and then the resultant was inserted into the Pvu II site of a pRS306 integration vector. The plasmid pRS-AtGFT1/VSVG was cleaved with EcoR V, and then the S. cerevisiae W303-1A strain was transformed. Transformation was carried out using the lithium acetate method. After transformation, the resultant was inoculated on an SD-Ura medium plate (2% glucose, 0.67% Yeast Nitrogen Base w/o amino acids (produced by Difco Laboratories), nucleic acid bases excluding uracil and an amino acid mixture (20 to 400 mg/l)) and then cultured at 30° C. for 2 days, thereby obtaining transformants. The transformants were scraped off the plate, and then subjected to a simple PCR method (whereby the transformants were suspended in a PCR reaction solution) so as to confirm incorporation onto the chromosome. The thus obtained transformants were determined to be of a YCH02 strain.

(3) Preparation of YCH03 Strain

A POFUT1 gene is located on human chromosome 20. The cDNA nucleotide sequence of the POFUT1 gene has been registered under AF375884 in a database. The full-length cDNA of the POFUT1 gene was amplified by PCR using primer E (AGAATTCATGGGCGCCGCCG: SEQ ID NO: 17) and primer F (GCTCCGGCTCGAGTCAGAACTCGTCCCGCA: SEQ ID NO: 18). A human brain-derived cDNA library (Clontech) was used as a template. The PCR product was cleaved at EcoR I and Xho I sites and then incorporated into the EcoR I/Xho I site of a yeast expression plasmid Yep352GAP, thereby constructing a plasmid YEp352GAP-POFUT1. Next, the 5′ region of POFUT1 was amplified using primer E and primer G (AGCCCGCGGGCATTGAGATCTGTACTAGTCCCGGGAGCGGCAGAAGCAGC: SEQ ID NO: 19). The DNA fragments were cleaved at EcoR I and Sac II sites and then replaced by the same sites of YEp352GAP-POFUT1. Furthermore, to Spe I and Bgl II sites newly added by this process, a DNA nucleotide sequence for expression of a FLAG antigen, which had been cleaved from a vector pESC-trp at the Spe I/Bgl II site, was inserted, thereby constructing a YEp352GAP-FLAG/POFUT1 plasmid. A region containing a yeast GAPDH promoter and terminator was excised with BamH I from the plasmid, both ends were blunted, and then the resultant was inserted into the Pvu II site of a pRS305 integration vector. The plasmid pRS-FLAG/POFUT1 was cleaved with EcoR V and then the S. cerevisiae W303-1B strain was transformed. Transformation was carried out using the lithium acetate method. After transformation, the resultant was inoculated on an SD-Leu medium plate (2% glucose, 0.67% Yeast Nitrogen Base w/o amino acids (produced by Difco Laboratories), nucleic acid bases excluding leucine and an amino acid mixture (20 to 400 mg/l)) and then cultured at 30° C. for 2 days, thereby obtaining transformants. The transformants were scraped off the plate and then subjected to a simple PCR method (whereby the transformants were suspended in a PCR reaction solution) so as to confirm incorporation onto the chromosome. The thus obtained transformants were determined to be of a YCH03 strain.

(4) Preparation of YCH04 Strain

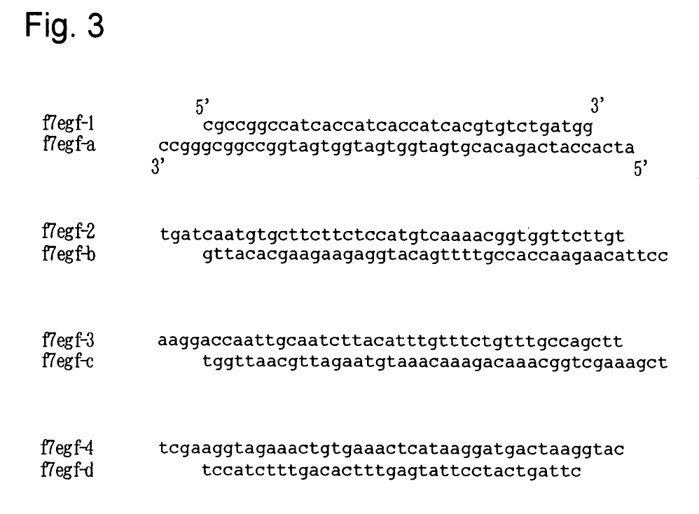

An F7 gene is located on human chromosome 13. The cDNA nucleotide sequence of the F7 gene has been registered under NM000131 in a database. The expression product of the F7 gene is a human coagulation factor (human factor VII). To cause expression and secretion of EGF domains (f7egf-1 to 4 and f7egf-a to f) in yeast, which are the endogenous domains in the protein, DNA nucleotide sequences encoding the amino acids are synthesized as shown in FIG. 3 (f7egf-1: SEQ ID NO: 25; f7egf-a: SEQ ID NO: 26; f7egf-2: SEQ ID NO: 27; f7egf-b: SEQ ID NO: 28; f7egf-3: SEQ ID NO: 29; f7egf-c: SEQ ID NO: 30; f7egf-4: SEQ ID NO: 31; and f7egf-d: SEQ ID NO: 32). Furthermore, these sequences were designed so that the His6 tag was fused to the 5′ side for purification. The 5′ sides of all single-stranded DNAs were phosphorylated and then 4 pairs of double-stranded DNAs were prepared by annealing reaction. The pairs were linked to each other, thereby preparing a DNA fragment having the Apa I site on the 5′ side and the Kpn I site on the 3′ side. The DNA fragment was inserted into the Apa I/Kpn I site of a pCR2.1 plasmid to confirm the nucleotide sequence. The resultant was cleaved at Nae I and Kpn I sites and incorporated into the Nae I/Kpn I site of a previously reported plasmid pAFF2, thereby constructing a yeast secretion plasmid YEp352GAP-alpha/HisEGF. A region containing a yeast GAPDH promoter and terminator was excised with BamH I from the plasmid and then the resultant was inserted into the BamH I site of a pJJ246 integration vector. The plasmid pJJ-alpha/HisEGF was cleaved with EcoR V and then the S. cerevisiae W303-1B strain was transformed. Transformation was carried out using the lithium acetate method. After transformation, the resultant was inoculated on an SD-Trp medium plate (2% glucose, 0.67% Yeast Nitrogen Base w/o amino acids (produced by Difco Laboratories), nucleic acid bases excluding tryptophan, and an amino acid mixture (20 to 400 mg/l)) and then cultured at 30° C. for 2 days, thereby obtaining transformants. The transformants were scraped off the plate and then subjected to a simple PCR method (whereby the transformants were suspended in a PCR reaction solution) so as to confirm incorporation onto the chromosome. The thus obtained transformants were determined to be of a YCH04 strain.

(5) Preparation of YCH05 Strain

A MUR1 gene is located on chromosome 3 of Arabidopsis thaliana. The cDNA nucleotide sequence of the MUR1 gene has been registered under AY084574 in a database. YEp352GAPII-MUR1-HA that had been previously reported as an expression plasmid for this gene in yeast was cleaved at the Pvu I site. A region containing the cDNA of MUR1 was then ligated to a region containing a selection marker of pRS303 cleaved at the Pvu I site, thereby constructing a plasmid pRS303-MUR1/HA for integration.

An AtFX gene is located on chromosome 1 of Arabidopsis thaliana. The cDNA nucleotide sequence of the AtFX gene has been registered under AB034806 in a database. A region containing a yeast GAPDH promoter and terminator was excised at the BamH I site from YEp352GAPII-AtFX-myc that had been previously reported as an expression plasmid for this gene in yeast. The resultant was blunted and then inserted into pRS303-MUR1/HA cleaved at the Nae I site, thereby constructing a plasmid pRS303-MUR1/HA-AtFX/myc for integration. The plasmid was cleaved at the Nhe I site and then the S. cerevisiae W303-1A strain was transformed. Transformation was carried out using the lithium acetate method. After transformation, the resultant was inoculated on SD-His medium plates (2% glucose, 0.67% Yeast Nitrogen Base w/o amino acids (produced by Difco Laboratories), nucleic acid bases excluding histidine, and an amino acid mixture (20 to 400 mg/l)) and then cultured at 30° C. for 2 days, thereby obtaining transformants. The transformants were scraped off the plate and then subjected to a simple PCR method (whereby the transformants were suspended in a PCR reaction solution) so as to confirm incorporation onto the chromosome. The thus obtained transformants were determined to be of a YCH05 strain.

Upon PCR carried out for confirming recombination on the chromosomes of the transformants obtained in (1) to (5) above, primer H (TTCCAAACTGGAACACT: SEQ ID NO: 20) and primer I (GCTACAGCAATTAATACTTGA:SEQ ID NO: 21) were used in the case of the S. cerevisiae YCH01 strain and YCH02 strain, primer H and primer J (GCATTAGCCCATTCTTCCATCAG: SEQ ID NO: 22) were used in the case of the S. cerevisiae YCH03 strain, primer K (GTAAAACGACGGCCAG: SEQ ID NO: 23) and primer F (GCTCCGGCTCGAGTCAGAACTCGTCCCGCA: SEQ ID NO: 18) were used in the case of the S. cerevisiae YCH04 strain, and primer H and primer L (GGCAATTTCTACTCGGGTTCAGC: SEQ ID NO: 24) were used in the case of the S. cerevisiae YCH05 strain. As a result, whereas nothing was amplified in the cases of parent strains (W303-1A and W303-1B strains), bands derived from the introduced plasmid DNA nucleotide sequences were amplified in the cases of the transformants.

(6) Preparation of SDP01 Strain

The AtGFT1 gene containing the VSVG portion was amplified by PCR using the plasmid pRS-AtGFT1/VSVG constructed in (2) above as a template, primer M (ACCTTCGAAACGATGTCGTCCTCTCGATTC: SEQ ID NO: 33), and primer N (ACCTCTAGATCATTTACCCAATCTATTC: SEQ ID NO: 34). The PCR product was cleaved at Nsp V and Xba I sites and then inserted into the Nsp V/Xba I site of the plasmid pPICZαA, thereby constructing a plasmid pPICZ-AtGFT1/VSVG. The MUR1 gene containing the HA portion was amplified by PCR using a plasmid YEp352GAPII-MUR1-HA as a template, primer O (TATGGTACCATGGCGTCAGAGAACAAC: SEQ ID NO: 35), and primer P (CTTGGGCCCTTAAGCCTTGGCAACGTG: SEQ ID NO: 36). The PCR product was cleaved at Kpn I and Apa I sites and then inserted into the Kpn I/Apa I site of a plasmid pGAPZαA, thereby constructing a plasmid pGAPZ-MUR1/HA. A DNA fragment containing GAP promoter, MUR1/HA, and terminator regions was excised at the Bgl II/Bam HI site from pGAPZ-MUR1/HA. The resultant was inserted into the Bam HI site of a plasmid pPICZ-AtGFT1/VSVG, thereby constructing a plasmid pPICZ-AtAtGFT1/VSVG-MUR1/HA for integration. The plasmid was cut open at the Avr II site and then transformed into the R pastoris SM 1168 strain. The transformants were inoculated on an YPAD plate containing Zeocin (100 mg/ml) and cultured at 30° C. for 5 days, thereby obtaining transformants of a Zeocin-resistant strain. The transformants were scraped off the plate and then subjected to a simple PCR method (whereby the transformants were suspended in a PCR reaction solution) so as to confirm incorporation onto the chromosome. The thus obtained transformants were determined to be of a SDP01 strain.

(7) Preparation of SDP02 Strain

POFUT1 containing a FLAG portion was amplified by PCR using the plasmid Yep352GAP-FLAG/POFUT1 constructed in (3) above as a template, primer Q (TAAGAATTCATGGGCGCCGCCG: SEQ ID NO: 37), and primer R (TAAGAATTCTCAGAACTCGTCCCGCA: SEQ ID NO: 38). The PCR product was cleaved with Eco RI and inserted into the Eco RI site of a plasmid pAO815, thereby constructing a plasmid pAO815-FLAG/POFUT1. The AtFX gene containing an myc portion was amplified by PCR using a plasmid YEp352GAPII-AtFX-myc as a template, primer S (CAACTCGAGATGTCTGACAAATCTGCC: SEQ ID NO: 39), and primer T (CTTGGGCCCTTAAGCCTTGGCAACGTG: SEQ ID NO: 40). The PCR product was cleaved at the Xho I/Apa I site and then inserted into the Xho I/Apa I site of a plasmid pGAPZα, thereby constructing a plasmid pGAPZ-AtFX/myc. A DNA fragment containing GAP promoter, AtFX/myc, and terminator regions was excised at the Bgl II/Bam HI site from the plasmid and then inserted into the Bam HI site of a plasmid PAO815-FLAG/POFUT1, thereby constructing a plasmid PAO815-FLAG/POFUT1-AtFX/myc for integration. The plasmid was cleaved at the Stu I site and then transformed into the P. pastoris SM 1168 strain. The transformants were inoculated on a histidine-deficient medium plate, cultured at 30° C. for 2 days, thereby obtaining transformants of a strain not requiring histidine. The transformants were scraped off the plate and then subjected to a simple PCR method (whereby the transformants were suspended in a PCR reaction solution) so as to confirm incorporation onto the chromosome. The thus obtained transformants were determined to be of a SDP02 strain.

(8) Preparation of SDP03 Strain

A F7 gene containing a His-tag portion was amplified by PCR using the plasmid YEp352GAP-alpha His/EGF constructed in (4) above as a template, primer U (TAATACGTACATCACCATCACCATCAC: SEQ ID NO: 41), and primer V (TAAGAATTCTTAGTCATCCTTATGAGTTTC: SEQ ID NO: 42). The PCR product was cleaved with Sna BI/Eco RI and then inserted into the Sna BI/Eco RI site of a plasmid pPIC9k, thereby constructing a plasmid pPIC9k-alpha His/EGF. The plasmid was cleaved at the Stu I site and then transformed into the P. pastoris SM 1168 strain. The transformants were inoculated on a histidine-deficient medium plate and cultured at 30° C. for 2 days, thereby obtaining transformants of a strain not requiring histidine. The transformants were scraped off the plate, and then subjected to a simple PCR method (whereby the transformants were suspended in a PCR reaction solution) so as to confirm incorporation onto the chromosome. The thus obtained transformants were determined to be of a SDP03 strain.

### EXAMPLE 2

### Preparation of Strains Wherein all the Genes Associated with the Synthesis System of O-Fucosylated Protein are Introduced

(1) Preparation of S. cerevisiae YCH09 Strain and YCH10 Strain

The transformed strains (the S. cerevisiae YCH01 strain was crossed with YCH03 strain, and the S. cerevisiae YCH02 strain was crossed with YCH03 strain) were each crossed on an YPAD medium plate. The thus crossed strains were each inoculated on an SD-Ura/Leu medium plate (2% glucose, 0.67% Yeast Nitrogen Base w/o amino acids (produced by Difco Laboratories), nucleic acid bases excluding uracil and leucine, and an amino acid mixture (20 to 400 mg/l)) and then cultured at 30° C. for 2 days, thereby selecting diploids. The thus obtained diploids were separated by tetrad analysis and then replicated on SD-Ura/Leu medium plates, thereby obtaining target transformants (the S. cerevisiae YCH06 strain and the S. cerevisiae YCH07 strain).

The transformed strains (the S. cerevisiae YCH04 strain and the YCH05 strain) were crossed on an YPAD medium plate. The thus crossed strains were inoculated on an SD-Trp/His medium plate (2% glucose, 0.67% Yeast Nitrogen Base w/o amino acids (produced by Difco Laboratories), nucleic acid bases excluding tryptophan and histidine, and an amino acid mixture (20 to 400 mg/l)) and then cultured at 30° C. for 2 days, thereby selecting diploids. The thus obtained diploids were separated by tetrad analysis and then replicated on an SD-Trp/His medium plate, thereby obtaining a target transformant, the S. cerevisiae YCH08 strain.

Furthermore, the transformed strains (the S. cerevisiae YCH06 strain was crossed with the YCH08 strain and the S. cerevisiae YCH07 strain was crossed with the YCH08 strain) were each crossed on an YPAD medium plate. The thus crossed strains were each inoculated on an SD-Ura/Leu/Trp/His medium plate (2% glucose, 0.67% Yeast Nitrogen Base w/o amino acids (produced by Difco Laboratories), nucleic acid bases excluding uracil, leucine, tryptophan, and histidine, and an amino acid mixture (20 to 400 mg/l)) and then cultured at 30° C. for 2 days, thereby selecting diploids. The thus obtained diploids were separated by tetrad analysis and then replicated on SD-Ura/Leu/Trp/His medium plates, thereby obtaining target transformants (the S. cerevisiae YCH09 strain and the S. cerevisiae YCH10 strain).

The S. cerevisiae YCH09 strain and the S. cerevisiae YCH10 strain were deposited with the International Patent Organism Depositary (IPOD) (Tsukuba Central 6, 1-1-3 Higashi, Tsukuba, Ibaraki, Japan), the National Institute of Advanced Industrial Science and Technology (AIST), on Mar. 24, 2005, under accession numbers FERM BP-10303 and FERM BP-10304, respectively.

(2) Preparation of P. pastoris SDP05 Strain

The transformed SDP01 strain was transformed with a plasmid PAO815-FLAG/POFUT1-AtFX/myc that had been cut open at the Stu I site. The transformants were inoculated on a histidine-deficient medium plate and then cultured at 30° C. for 2 days, thereby obtaining transformants of a strain not requiring histidine. The transformants were scraped off the plate and then subjected to a simple PCR method (whereby the transformants were suspended in a PCR reaction solution) so as to confirm incorporation onto the chromosome. The thus obtained transformants were determined to be of a SDP04 strain. Furthermore, the SDP04 strain was transformed with a plasmid pPIC9k-alpha His/EGF that had been cleaved at the Stu I site. The transformants were inoculated on an YPDA plate containing Geneticin (200 μg/ml) and then cultured at 30° C. for 5 days, thereby obtaining transformants of a kanamycin-resistant strain. The transformants were scraped off the plate and then subjected to a simple PCR method (whereby the transformants were suspended in a PCR reaction solution) so as to confirm incorporation onto the chromosome. The thus obtained transformants were determined to be of a SDP05 strain.

### EXAMPLE 3

### Confirmation of Gene Expression

(1) Confirmation of Gene Expression in the S. cerevisiae YCH09 Strain and YCH10 Strain

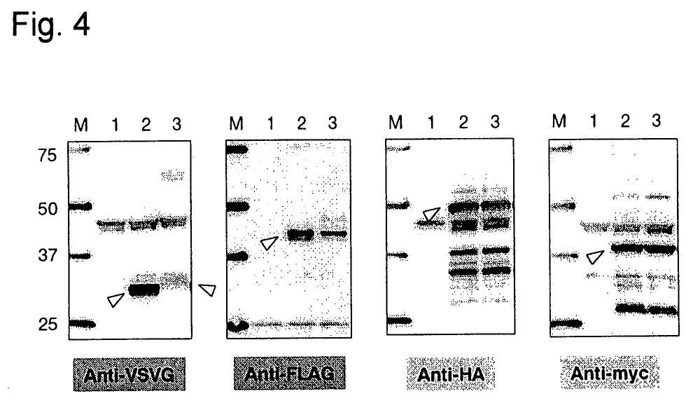

The S. cerevisiae YCH09 strain and the S. cerevisiae YCH10 strain prepared in Example 2 were cultured in 3 ml of an YPAD medium (2% polypeptone, 1% yeast extract, 2% glucose, and adenine (40 mg/l)) at 30° C. for 12 hours. Microbial bodies were collected by centrifugation. The microbial bodies were disrupted using glass beads. After solubilizing insoluble protein by adding a surfactant to a disruption solution, a supernatant was obtained by centrifugation. The obtained supernatant, which was a crude enzyme solution, was denatured using an SDS sample buffer, and then subjected to Western blot analysis according to a conventional method. In Western blot analysis, a goat anti-VSVG antibody, a mouse anti-HA antibody, a mouse anti-myc antibody, and a mouse anti-FLAG antibody were used as primary antibodies. When the goat anti-VSVG antibody was used as a primary antibody, an anti-goat Ig antibody horseradish peroxidase complex was used as a secondary antibody. When the mouse anti-HA antibody, the mouse anti-myc antibody, and the mouse anti-FLAG antibody were used as primary antibodies, the anti-mouse Ig antibody horseradish peroxidase complex was used as a secondary antibody for each case. Detection was carried out using an ECL plus system (Amersham Biosciences K.K.) and a chemiluminescence detector (FUJI PHOTO FILM CO., LTD.). The results are shown in FIG. 4.

As shown in FIG. 4, whereas no signals were observed in the case of a W303-1A strain that was a wild strain (control strain), in the case of transformed strains (the YCH09 strain and the YCH10 strain), signals were confirmed at each molecular weight position corresponding to an hGFT gene product or an AtGFT1 gene product by the use of the anti-VSVG antibody, a MUR1 gene product by the use of the anti-HA antibody, an AtFX gene product by the use of the anti-myc antibody, or a POFUT1 gene product by the use of the anti-FLAG antibody.

(2) Confirmation of Gene Expression in P. pastoris SDP05 Strain

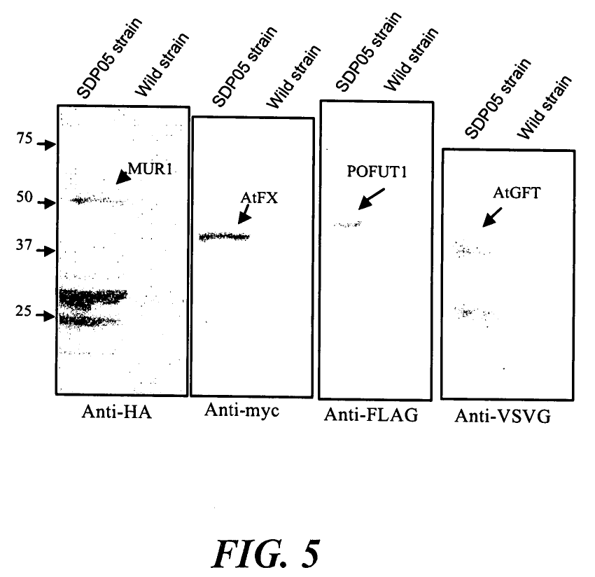

Similarly, the P. pastoris SDP05 strain prepared in Example 2 was cultured in 50 ml of BMMY (2% polypeptone, 1% yeast extract, 1.34% yeast nitrogen base, 0.3 M potassium chloride, and 0.1 M phosphate buffer (pH6.0)) at 30° C. for 24 hours. 1% methanol was added every 24 hours, so as to induce protein expression. 72 hours later, culture was stopped and then microibal bodies were collected by centrifugation. Protein was extracted from the collected microbial bodies using a Y-PER Yeast Protein Extraction Reagent (Pierce Biotechnology, Inc.). Samples were denatured using an SDS sample buffer, and then subjected to Western blot analysis according to a conventional method. In Western blot analysis, a goat anti-VSVG antibody, a mouse anti-HA antibody, a mouse anti-myc antibody, and a mouse anti-FLAG antibody were used as primary antibodies. When the goat anti-VSVG antibody was used as a primary antibody, an anti-goat Ig antibody horseradish peroxidase complex was used as a secondary antibody. When the mouse anti-HA antibody, the mouse anti-myc antibody, and the mouse anti-FLAG antibody were used as primary antibodies, the anti-mouse Ig antibody horseradish peroxidase complex was used as a secondary antibody for each case. Detection was carried out using an ECL plus system (Amersham Biosciences K.K.) and a chemiluminescence detector (FUJI PHOTO FILM CO., LTD.). The results are shown in FIG. 5.

As shown in FIG. 5, no signals were observed at all in the case of the P. pastoris SM 1168 strain that was a wild strain (control strain). However, in the case of the transformed strain (SDP05 strain), signals were confirmed at each molecular weight position corresponding to an AtGFT1 gene product by the use of the anti-VSVG antibody, a MUR1 gene product by the use of the anti-HA antibody, an AtFX gene product by the use of the anti-myc antibody, or a POFUT1 gene product by the use of the anti-FLAG antibody.

### EXAMPLE 4

### Purification and Analysis of EGF Domains

(1) EGF Domains Derived from the Culture Product of the S. cerevisiae YCH09 Strain and YCH10 Strain

The S. cerevisiae YCH09 strain and the S. cerevisiae YCH10 strain were cultured in 30 ml of an YPAD medium (2% polypeptone, 1% yeast extract, 2% glucose, adenine (40 mg/l)) at 30° C. for 60 hours. Culture supernatants were then obtained by centrifugation. The S. cerevisiae YCH04 strain was similarly cultured as a control strain and then a culture supernatant was obtained. After adjustment of pH of the culture supernatants at pH 8.0, Ni-NTA agarose (QIAGEN) was added to the culture supernatants. The resultants were washed with a phosphate buffer (pH 8.0) containing 0.3 M NaCl and 20 mM imidazole and then eluted using a phosphate buffer (pH 8.0) containing 0.3 M NaCl and 250 mM imidazole. The thus obtained eluates were determined to be purified EGF domain specimens.

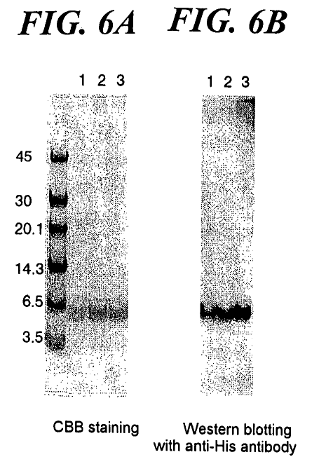

The presence of the purified EGF domains was confirmed by SDS-PAGE using a tris-tricine buffer system and then CBB staining. As shown in FIG. 6 (A), as a result of cleavage off of the prepro sequence of an α-factor by Kex2 protease within yeast cells, a band was detected at an approximately 5.7 kDa position corresponding to the size of the EGF domain secreted in media. Furthermore, no signals were observed in the case of the specimen derived from the culture supernatant of the W303-1A strain that was a wild strain (control strain).

Furthermore, Western blot analysis was carried out according to a conventional method. An anti-His5 antibody horseradish peroxidase complex was used as a primary antibody. Detection was carried out using an ECL plus system (Amersham Biosciences K.K.) and a chemiluminescence detector (FUJI PHOTO FILM CO., LTD.). The results are shown in FIG. 6 (B). Regarding specimens for which bands had been detected by CBB staining, signals were confirmed at positions where bands had been observed by CBB staining in all the specimens including the specimen derived from the control strain (YCH04 strain) that had been caused to express only EGF domains.

When the purified EGF domains were subjected to N-terminal amino acid sequence analysis, the obtained domains were found to have sequences as designed, from which the prepro sequence of an α-factor had been cleaved off.

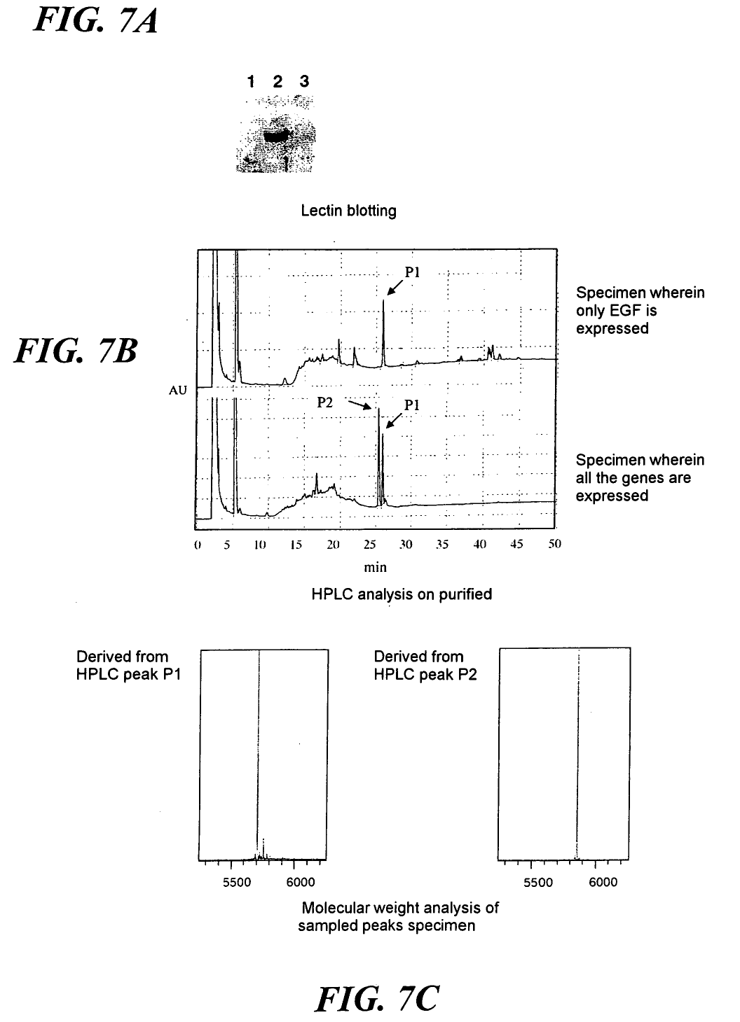

The thus obtained purified EGF domain specimens were subjected to SDS-PAGE according to a conventional method and then to lectin blotting on PVDF membranes. As a lectin, a biotin-labeled lectin (Honen Corporation) derived from Aleuria aurantia, which was a fucose-recognizing lectin, was used. Subsequently, an avidin-horseradish peroxidase complex was used. Detection was carried out using an ECL plus system (Amersham Biosciences K.K.) and a chemiluminescence detector (FUJI PHOTO FILM CO., LTD.). The results are shown in FIG. 7(A).

No signals were observed in the case of a specimen derived from the culture supernatant of the control strain (the YCH04 strain) caused to express only EGF domains. On the other hand, in the case of specimens derived from the transformed strains (the YCH09 strain and the YCH10 strain) caused to express all the genes, signals due to lectin were confirmed at positions where bands had been observed by CBB staining. In particular, stronger signals were detected in the case of a specimen derived from the strain (the YCH10 strain) wherein a novel AtGFT1, a GDP-fucose transporter, had been introduced.

To quantify the amount of linked fucose in the thus obtained EGF domain specimens, a purity test was conducted by HPLC analysis using a reverse phase column. A cosmocil C4 butyl column (4.6×150 mm) was used as a column. 0.1% trifluoroacetic acid (solvent I) and 0.1% trifluoroacetic acid-containing acetonitrile (solvent II) were used as solvents. The column was equilibrated by previously causing solvent I to flow at a flow rate of 1.0 ml/min. The proportion of solvent II was linearly increased to 70% for 50 minutes immediately after injection of a sample, thereby eluting EGF domains. Detection was carried out using an UV detector (detection wavelength of 215 nm). The results are shown in FIG. 7 (B). First, the specimen derived from the control strain (the YCH04 strain) expressing only EGF domains was eluted as single peak P1 at around 26 minutes. In contrast, in the case of the specimen for which signals had been detected by lectin blotting, a new peak P2 was eluted immediately before a control peak together with peak P1. The area ratio of P1:P2 is approximately 4:6. To verify whether or not this indicates EGF domains having the O-fucose structure, each peak was sampled.

EGF domain specimens sampled from HPLC were each freeze-dried and then the products were subjected to molecular weight measurement by MALDI-TOF-MS. The results are shown in FIG. 7(C). A peak indicating a molecular weight of approximately 5704 was detected for the specimen derived from peak P1 in HPLC and a peak indicating the same of approximately 5850 was detected for the specimen derived from peak P2 in HPLC. These molecular weights are in agreement with the theoretical values of the designed EGF domains. It is known that unlike other hexoses, oxygen at position 6 of fucose is deoxygenated, so that its increase in molecular weight is as low as 146 compared with that of a general hexose. Moreover, the molecular weight of peak P1 indicates the presence of disulfide bonds at 3 positions in an EGF domain. Accordingly, linkage of one fucose molecule in an EGF domain forming an appropriate folding structure was shown.

(2) EGF Domain Derived from the Culture Product of the P. pastoris SDP05 Strain

The P. pastoris SDP05 strain was cultured in 100 ml of BMGY (2% polypeptone, 1% yeast extract, 1.34% yeast nitrogen base, 1% glycerol, 0.3 M potassium chloride, and 0.1 M phosphate buffer (pH 6.0)) at 30° C. for 24 hours. 1% methanol was added every 24 hours so as to induce protein expression. 72 hours later, culture was stopped and then a culture supernatant was obtained by centrifugation. Furthermore, the P. pastoris SM 1168 strain was similarly cultured as a control strain and then a culture supernatant was obtained. The thus collected culture supernatants were subjected to an His Trap Chelating column (Pharmacia K.K.) and then washed with a phosphate buffer (pH 7.0) containing 0.3 M NaCl and 20 mM imidazole, followed by elution using a phosphate buffer (pH 7.0) containing 0.3 M NaCl and 300 mM imidazole. The thus eluted protein peaks were collected and determined to be purified EGF domain specimens. In addition, purification was carried out using AKTA 100-S produced by Pharmacia K.K.

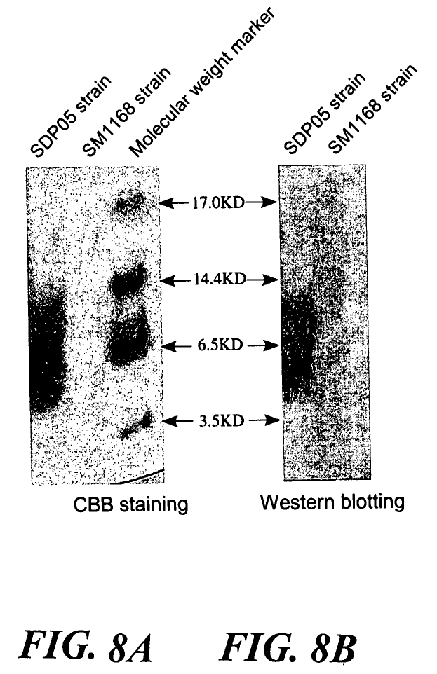

The presence of purified EGF domains was confirmed by SDS-PAGE using a tris-tricine buffer system and then by CBB staining. As shown in FIG. 8 (A), a band was detected at approximately the 5.7 kDa position corresponding to the size of the EGF domain that had been secreted in the medium after cleavage off of the prepro sequence of an α-factor by Kex2 protease in yeast cells. Furthermore, no signals were observed in the case of the specimen derived from the culture supernatant of an SM 1168 strain that was a wild strain (control strain).

Furthermore, Western blot analysis was carried out according to a conventional method. An anti-His5 antibody horseradish peroxidase complex was used as a primary antibody. Detection was carried out using an ECL plus system (Amersham Biosciences K.K.) and a chemiluminescence detector (FUJI PHOTO FILM CO., LTD.). The results are shown in FIG. 8 (B).

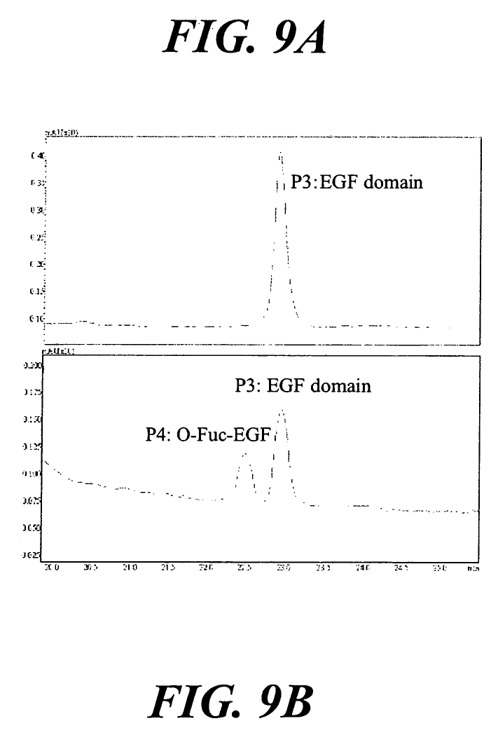

To quantify the amount of linked fucose in the EGF domain specimens obtained from P. pastoris, a purity test was conducted by HPLC analysis using a reverse phase column. A cosmocil C4 butyl column (4.6×150 mm) was used as a column. 0.1% trifluoroacetic acid (solvent I) and 0.1% trifluoroacetic acid-containing acetonitrile (solvent II) were used as solvents. The column was equilibrated by previously causing solvent I to flow at a flow rate of 1.0 ml/min. The proportion of solvent II was linearly increased to 70% for 50 minutes immediately after injection of a sample, thereby eluting EGF domains. Detection was carried out using an UV detector (detection wavelength of 215 nm). The results are shown in FIG. 9. The EGF domain specimen obtained from the control strain (SDP03 strain) caused to express only EGF was eluted as single peak P3 at around 23 minutes. This was the same as that of a standard product derived from the control strain (the YCH04) expressing only EGF domains in S. cerevisiae strain. Furthermore, in the case of the EGF domain specimen obtained from the SDP05 strain, a new peak P4 was eluted immediate before the 23-minute peak together with peak P3. The area ratio of P3:P4 is approximately 2:1, indicating that the EGF domain has the O-fucose structure.

All publications, patents, and patent applications cited herein are incorporated herein by reference in their entirety.

### INDUSTRIAL APPLICABILITY

According to the present invention, a yeast transformant wherein genes associated with the synthesis system of O-fucosylated protein, which are originally absent in yeast, are introduced is provided. The use of such a yeast transformant enables production of O-fucosylated protein in large quantities.

Furthermore, a yeast transformant wherein some of the genes associated with the synthesis system of O-fucosylated protein of the present invention are not introduced can be utilized as a material for screening for genes associated with the synthesis system of O-fucosylated protein or the proteins expressed by the genes in animals or plants.

Hence, such a yeast transformant of the present invention is useful as a means for synthesizing various glycostructures based on that of O-fucosylated protein and for screening for novel useful genes associated with the synthesis system of O-fucosylated protein or for the proteins expressed by such genes. For example, such a transformant can greatly contribute to the elucidation of causes of diseases associated with the structures of sugar chains of glycoproteins, or the like. It can be used for developing therapeutic methods therefor, pharmaceutical products, and the like.

Furthermore, a metabolic system that is originally absent in yeast is used in the present invention, so that there is an advantage in that high-density culture of transformants can be expected without affecting the synthesis system of yeast endogenous glycoprotein.

What is claimed is:

1. A yeast transformant, wherein genes associated with a synthesis system of O-fucosylated protein are introduced.

2. The yeast transformant of claim 1, wherein the genes associated with the synthesis system of O-fucosylated protein are a GDP-fucose synthase gene, a GDP-fucose transporter gene, a fucosyltransferase gene, and a fucose receptor gene.

3. The yeast transformant of claim 2, wherein the fucose receptor gene has a secretion signal sequence added thereto.

4. The yeast transformant of claim 3, wherein the fucose receptor gene is a DNA containing a nucleotide sequence encoding at least an EGF domain.

5. A recombinant vector, wherein a GDP-fucose transporter gene is inserted under control of a promoter for yeast.

6. A recombinant vector, wherein a fucosyltransferase gene is inserted under control of a promoter for yeast.

7. A recombinant vector, wherein a fucose receptor gene is inserted under control of a promoter for yeast.

8. The recombinant vector of claim 7, wherein the fucose receptor gene has a secretion signal sequence added thereto.

9. The recombinant vector of claim 8, wherein the fucose receptor gene is a DNA containing a nucleotide sequence encoding at least an EGF domain.

10. A recombinant vector, wherein a GDP-fucose synthase gene, a GDP-fucose transporter gene and/or a fucosyltransferase gene are inserted under control of a promoter for yeast.

11. A use of the recombinant vector of any one of claims 5 to 10 for preparing the yeast transformant of any one of claims 1 to 4.

12. A method for producing O-fucosylated protein, which comprises culturing the yeast transformant of any one of claims 1 to 4 and collecting O-fucosylated protein from the culture product.

13. A gene kit for preparing the yeast transformant of any one of claims 1 to 4, which contains a GDP-fucose synthase gene, a GDP-fucose transporter gene, a fucosyltransferase gene, and a gene encoding a fucose receptor.

14. A yeast transformant, wherein the following genes (a) or (b) are introduced:
(a) a GDP-fucose synthase gene and a GDP-fucose transporter gene; or
(b) a GDP-fucose synthase gene, a fucosyltransferase gene, and/or a fucose receptor gene.

15. A method for screening for genes associated with the synthesis system of O-fucosylated protein and/or the proteins expressed the genes, which comprises culturing the yeast transformant of claim 14, collecting the expressed proteins, and screening for the genes and/or the proteins using the presence of sugar molecules in the expressed proteins as an indicator.

16. A method for confirming or measuring the activity of proteins expressed by genes associated with the synthesis system of O-fucosylated protein, which comprises culturing the yeast transformant of claim 14, collecting the expressed proteins, and confirming or measuring the activity using the presence of sugar molecules in the expressed proteins as an indicator.

17. A gene kit for preparing the yeast transformant of claim 14, which contains the following genes (a) or (b):
(a) a GDP-fucose synthase gene and a GDP-fucose transporter gene; or
(b) a GDP-fucose synthase gene, a fucosyltransferase gene, and/or a fucose receptor gene.

18. A method for measuring the number of disulfide bonds in protein, which comprises culturing the yeast transformant of any one of claims 1 to 4, collecting O-fucosylated protein from the culture product, and measuring the molecular weights of the obtained O-fucosylated protein and protein with no fucose linked thereto to find an increase in molecular weight as a result of the linkage of fucose to protein.

19. A protein, which has the amino acid sequence represented by SEQ ID NO: 8 in the sequence listing or an amino acid sequence derived from such amino acid sequence by deletion, substitution, or addition of 1 or several amino acids, and which has GDP-fucose transporter activity.

20. A gene, which encodes the protein of claim 19.


SEQUENCE LISTING (42 sequences)

SEQ ID NO: 1 - DNA, 1122 bp, Arabidopsis thaliana
  CDS (1)..(1119): MUR1
atggcgtcagagaacaacggatccagatccgattccgaatccatcaccgctcccaaagctgattccaccgtcgttgaaccgaggaagatagctctgatcaccggaatcaccggccaggacggatcatacctgacggagttccttctcggaaaaggctacgaatttcatggtctgatccgtcgatcatcgaatttcaacacccagcgaatcaaccatatctacatcgatccacacaatgtcaacaaagctctgatgaaactccactacgccgatctcaccgacgcttcctctctccgtcgttggatcgatgtgatcaagcctgacgaagtttataacctagctgctcaatctcacgtcgctgtctccttcgagatccctgattacacagccgatgtagtcgcaaccggtgctctccgtctccttgaagccgtcagatctcacaccatcgacagtggccgtaccgtcaagtattaccaagccggatcttcggagatgtttggatcaactcctcctccacaatcggagacgacgccgtttcaccccagatctccttacgcagcttccaaatgcgctgctcattggtacacagtgaattacagagaggcgtacggtctcttcgcttgtaacggaatcttgttcaatcacgagtcacctcgccgtggtgagaatttcgtgacgaggaagatcacaagagcattgggaaggatcaaggttggtttgcagacgaagctattccttgggaatttgcaagcgtcaagagattggggatttgcaggagattatgtggaagcaatgtggttgatgttgcagcaagagaagccagatgattacgttgtggcaacagaggaaggacacacagtggaagagtttcttgatgtgtcatttgggtatttgggactcaattggaaagattatgttgagattgaccagaggtactttaggcctgctgaagtagataaccttcaaggagatgcaagcaaggcaaaggaagtgttggggtggaaaccacaagtagggtttgagaagcttgtgaagatgatggttgatgaagatcttgagcttgctaagagggagaaagtgcttgttgatgctggatacatggatgctaagcagcaaccttga

SEQ ID NO: 2 - protein, 373 aa, Arabidopsis thaliana
MASENNGSRSDSESITAPKADSTVVEPRKIALITGITGQDGSYLTEFLLGKGYEFHGLIRRSSNFNTQRINHIYIDPHNVNKALMKLHYADLTDASSLRRWIDVIKPDEVYNLAAQSHVAVSFEIPDYTADVVATGALRLLEAVRSHTIDSGRTVKYYQAGSSEMFGSTPPPQSETTPFHPRSPYAASKCAAHWYTVNYREAYGLFACNGILFNHESPRRGENFVTRKITRALGRIKVGLQTKLFLGNLQASRDWGFAGDYVEAMWLMLQQEKPDDYVVATEEGHTVEEFLDVSFGYLGLNWKDYVEIDQRYFRPAEVDNLQGDASKAKEVLGWKPQVGFEKLVKMMVDEDLELAKREKVLVDAGYMDAKQQP

SEQ ID NO: 3 - DNA, 939 bp, Arabidopsis thaliana
  CDS (1)..(936): AtFX
atgtctgacaaatctgccaaaatcttcgtcgcgggtcatcgtggtttggttggatctgccattgtccgcaagcttcaggaacaaggtttcaccaatctcgttcttaaaacacacgccgagcttgatctcactcgtcaagccgatgttgaatccttcttttctcaagagaagccagtttatgtaatcctagcagcagctaaagttggtggtattcacgctaacaacacctatcctgctgatttcattggtgtcaatctccagattcagaccaatgtgatccactctgcatatgagcacggtgtgaagaagcttctcttccttggatcatcctgcatttaccctaaatttgctcctcagccaattcctgagtctgctttgttaacagcatcgcttgaaccaactaatgagtggtatgctattgctaagatcgctgggattaagacttgtcaggcttataggattcagcacggatgggatgcaatctctggcatgcctactaatctctatggtcctaatgacaatttccacccggagaattctcatgtgcttcctgctcttatgaggaggttccacgaggcgaaagtgaatggagcggaggaagttgtggtgtggggtacaggtagtccgttgagggagttcttgcatgttgatgatttggctgatgcttgtgttttcttgctggatcgatacagcgggttggagcatgttaacattggaagtggtcaagaagtgactattagagagttggctgagttggtgaaagaggttgttggttttgaagggaagcttggatgggattgcactaagccagatggcacaccgaggaaacttatggacagctcaaagctcgcgtctttgggttggacacctaaggtttctcttagagatggtctgagccaaacttatgattggtatttgaagaatgtttgcaaccgataa

SEQ ID NO: 4 - protein, 312 aa, Arabidopsis thaliana
MSDKSAKIFVAGHRGLVGSAIVRKLQEQGFTNLVLKTHAELDLTRQADVESFFSQEKPVYVILAAAKVGGIHANNTYPADFIGVNLQIQTNVIHSAYEHGVKKLLFLGSSCIYPKFAPQPIPESALLTASLEPTNEWYAIAKIAGIKTCQAYRIQHGWDAISGMPTNLYGPNDNFHPENSHVLPALMRRFHEAKVNGAEEVVVWGTGSPLREFLHVDDLADACVFLLDRYSGLEHVNIGSGQEVTIRELAELVKEVVGFEGKLGWDCTKPDGTPRKLMDSSKLASLGWTPKVSLRDGLSQTYDWYLKNVCNR

SEQ ID NO: 5 - DNA, 1095 bp, human
  CDS (1)..(1092): hGFT
atgaatagggcccctctgaagcggtccaggatcctgcacatggcgctgaccggggcctcagacccctctgcagaggcagaggccaacggggagaagccctttctgctgcgggcattgcagatcgcgctggtggtctccctctactgggtcacctccatctccatggtgttccttaataagtacctgctggacagcccctccctgcggctggacacccccatcttcgtcaccttctaccagtgcctggtgaccacgctgctgtgcaaaggcctcagcgctctggccgcctgctgccctggtgccgtggacttccccagcttgcgcctggacctcagggtggcccgcagcgtcctgcccctgtcggtggtcttcatcggcatgatcaccttcaataacctctgcctcaagtacgtcggtgtggccttctacaatgtgggccgctcactcaccaccgtcttcaacgtgctgctctcctacctgctgctcaagcagaccacctccttctatgccctgctcacctgcggtatcatcatcgggggcttctggcttggtgtggaccaggagggggcagaaggcaccctgtcgtggctgggcaccgtcttcggcgtgctggctagcctctgtgtctcgctcaacgccatctacaccacgaaggtgctcccggcggtggacggcagcatctggcgcctgactttctacaacaacgtcaacgcctgcatcctcttcctgcccctgctcctgctgctcggggagcttcaggccctgcgtgactttgcccagctgggcagtgcccacttctgggggatgatgacgctgggcggcctgtttggctttgccatcggctacgtgacaggactgcagatcaagttcaccagtccgctgacccacaatgtgtcgggcacggccaaggcctgtgcccagacagtgctggccgtgctctactacgaggagaccaagagcttcctctggtggacgagcaacatgatggtgctgggcggctcctccgcctacacctgggtcaggggctgggagatgaagaagactccggaggagcccagccccaaagacagcgagaagagcgccatgggggtgtga

SEQ ID NO: 6 - protein, 364 aa, human
MNRAPLKRSRILHMALTGASDPSAEAEANGEKPFLLRALQIALVVSLYWVTSISMVFLNKYLLDSPSLRLDTPIFVTFYQCLVTTLLCKGLSALAACCPGAVDFPSLRLDLRVARSVLPLSVVFIGMITFNNLCLKYVGVAFYNVGRSLTTVFNVLLSYLLLKQTTSFYALLTCGIIIGGFWLGVDQEGAEGTLSWLGTVFGVLASLCVSLNAIYTTKVLPAVDGSIWRLTFYNNVNACILFLPLLLLLGELQALRDFAQLGSAHFWGMMTLGGLFGFAIGYVTGLQIKFTSPLTHNVSGTAKACAQTVLAVLYYEETKSFLWWTSNMMVLGGSSAYTWVRGWEMKKTPEEPSPKDSEKSAMGV

SEQ ID NO: 7 - DNA, 1026 bp, Arabidopsis thaliana
  CDS (1)..(1023): AtGFT1
atgtcgtcctctcgattcgattcaaacaaacaattaacaacaagcagtctcgtaatcggctacgcactctgctcaagcctcctcgccgtaatcaacaaattagcaatcacatacttcaactacccaggtctcctcacagctcttcaatacctaacctgcaccgtcgccgtctatcttctcggcaaatcagggttaatcaatcacgatccattcacatgggacactgctaagaaatttctccctgcagctatcgtcttctacctcgctatcttcacaaacacaaacctcctccgtcacgctaatgtcgatactttcatcgtcttccgatctctaacaccgctcttagtcgcgatcgctgatactgtgtttcgtagtcagcctttaccttcgagacttacgtttttgtctctcgtcgttatcctcgctggagctgttgggtatgttgcaactgactcgtcttttactctaacggcttattcatgggctttggcttatcttgttaccattactactgaaatggtttacattaagcatatggtctctaatattaaattgaacatttggggattggttctctacaataatctactgtctctgatgatagcaccggtgttctggtttcttactggagaattcaccgaggttttcgcggcgttgagtgagaatagagggaatttgtttgagccttatgcgttttcttctgtggctgcgtcgtgtgtgtttggtttccttatcagttactttggatttgctgctaggaatgctatctctgctactgcttttacagtcactggtgttgttaacaagttcttgactgttgtcatcaatgttttgatttgggataagcatgccactcctgttggtttggtttgtcttctttttactatctgtggaggtgttggttatcagcagagtgtcaaattggataaacccattgagaaggtcagcgagaaagatagcgagaagggagaggaagatgaggagttgacgcagttggttccggggaaactagcttctgttgtatga

SEQ ID NO: 8 - protein, 341 aa, Arabidopsis thaliana
MSSSRFDSNKQLTTSSLVIGYALCSSLLAVINKLAITYFNYPGLLTALQYLTCTVAVYLLGKSGLINHDPFTWDTAKKFLPAAIVFYLAIFTNTNLLRHANVDTFIVFRSLTPLLVAIADTVFRSQPLPSRLTFLSLVVILAGAVGYVATDSSFTLTAYSWALAYLVTITTEMVYIKHMVSNIKLNIWGLVLYNNLLSLMIAPVFWFLTGEFTEVFAALSENRGNLFEPYAFSSVAASCVFGFLISYFGFAARNAISATAFTVTGVVNKFLTVVINVLIWDKHATPVGLVCLLFTICGGVGYQQSVKLDKPIEKVSEKDSEKGEEDEELTQLVPGKLASVV

SEQ ID NO: 9 - DNA, 1167 bp, human
  CDS (1)..(1164): POFUT1
atgggcgccgccgcgtgggcacggccgctgagcgtgtctttcctgctgctgcttctgccgctcccggggatgcccgcgggctcctgggacccggccggttacctgctctactgcccctgcatggggcgctttgggaaccaggccgatcacttcttgggctctctggcatttgcaaagctgctaaaccgtaccttggctgtccctccttggattgagtaccagcatcacaagcctcctttcaccaacctccatgtgtcctaccagaagtacttcaagctggagcccctccaggcttaccatcgggtcatcagcttggaggatttcatggagaagctggcacccacccactggccccctgagaagcgggtggcatactgctttgaggtggcagcccagcgaagcccagataagaagacgtgccccatgaaggaaggaaacccctttggcccattctgggatcagtttcatgtgagtttcaacaagtcggagctttttacaggcatttccttcagtgcttcctacagagaacaatggagccagagattttctccaaaggaacatccggtgcttgccctgccaggagccccagcccagttccccgtcctagaggaacacaggccactacagaagtacatggtatggtcagacgaaatggtgaagacgggagaggcccagattcatgcccaccttgtccggccctatgtgggcattcatctgcgcattggctctgactggaagaacgcctgtgccatgctgaaggacgggactgcaggctcgcacttcatggcctctccgcagtgtgtgggctacagccgcagcacagcggcccccctcacgatgactatgtgcctgcctgacctgaaggagatccagagggctgtgaagctctgggtgaggtcgctggatgcccagtcggtctacgttgctactgattccgagagttatgtgcctgagctccaacagctcttcaaagggaaggtgaaggtggtgagcctgaagcctgaggtggcccaggtcgacctgtacatcctcggccaagccgaccactttattggcaactgtgtctcctccttcactgcctttgtgaagcgggagcgggacctccaggggaggccgtcttctttcttcggcatggacaggccccctaagctgcgggacgagttctga

SEQ ID NO: 10 - protein, 388 aa, human
MGAAAWARPLSVSFLLLLLPLPGMPAGSWDPAGYLLYCPCMGRFGNQADHFLGSLAFAKLLNRTLAVPPWIEYQHHKPPFTNLHVSYQKYFKLEPLQAYHRVISLEDFMEKLAPTHWPPEKRVAYCFEVAAQRSPDKKTCPMKEGNPFGPFWDQFHVSFNKSELFTGISFSASYREQWSQRFSPKEHPVLALPGAPAQFPVLEEHRPLQKYMVWSDEMVKTGEAQIHAHLVRPYVGIHLRIGSDWKNACAMLKDGTAGSHFMASPQCVGYSRSTAAPLTMTMCLPDLKEIQRAVKLWVRSLDAQSVYVATDSESYVPELQQLFKGKVKVVSLKPEVAQVDLYILGQADHFIGNCVSSFTAFVKRERDLQGRPSSFFGMDRPPKLRDEF

SEQ ID NO: 11 - DNA, 414 bp, Artificial Sequence
  EGF domain added alpha-facter and His-tag
atgagatttccttcaatttttactgcagttttattcgcagcatcctccgcattagctgctccagtcaacactacaacagaagatgaaacggcacaaattccggctgaagctgtcatcggttacttagatttagaaggggatttcgatgttgctgttttgccattttccaacagcacaaataacgggttattgtttataaatactactattgccagcattgctgctaaagaagaaggggtatctttggataaaagagccggccatcaccatcaccatcacgtgtctgatggtgatcaatgtgcttcttctccatgtcaaaacggtggttcttgtaaggaccaattgcaatcttacatttgtttctgtttgccagctttcgaaggtagaaactgtgaaactcataaggatgactaa

SEQ ID NO: 12 - protein, 137 aa, Artificial Sequence
  EGF domain added alpha-facter and His-tag
MRFPSIFTAVLFAASSALAAPVNTTTEDETAQIPAEAVIGYLDLEGDFDVAVLPFSNSTNNGLLFINTTIASIAAKEEGVSLDKRAGHHHHHHVSDGDQCASSPCQNGGSCKDQLQSYICFCLPAFEGRNCETHKDD

SEQ ID NO: 13 - DNA, 30 bp, Artificial Sequence
  primerA
aatgagctcatgaatagggcccctctgaag

SEQ ID NO: 14 - DNA, 63 bp, Artificial Sequence
  primerB
actctagatcatttacccaatctattcatttcaatatcagtgtacacccccatggcgctcttc

SEQ ID NO: 15 - DNA, 28 bp, Artificial Sequence
  primerC
agagctcatgtcgtcctctcgattcgat

SEQ ID NO: 16 - DNA, 63 bp, Artificial Sequence
  primerD
cctctagatcatttacccaatctattcatttcaatatcagtgtatacaacagaagctagtttc

SEQ ID NO: 17 - DNA, 20 bp, Artificial Sequence
  primerE
agaattcatgggcgccgccg

SEQ ID NO: 18 - DNA, 30 bp, Artificial Sequence
  primerF
gctccggctcgagtcagaactcgtcccgca

SEQ ID NO: 19 - DNA, 50 bp, Artificial Sequence
  primerG
agcccgcgggcattgagatctgtactagtcccgggagcggcagaagcagc

SEQ ID NO: 20 - DNA, 17 bp, Artificial Sequence
  primerH
ttccaaactggaacact

SEQ ID NO: 21 - DNA, 21 bp, Artificial Sequence
  primerI
gctacagcaattaatacttga

SEQ ID NO: 22 - DNA, 23 bp, Artificial Sequence
  primerJ
gcattagcccattcttccatcag

SEQ ID NO: 23 - DNA, 16 bp, Artificial Sequence
  primerK
gtaaaacgacggccag

SEQ ID NO: 24 - DNA, 23 bp, Artificial Sequence
  primerL
ggcaatttctactcgggttcagc

SEQ ID NO: 25 - DNA, 36 bp, Artificial Sequence
  EGFdomain
cgccggccatcaccatcaccatcacgtgtctgatgg

SEQ ID NO: 26 - DNA, 44 bp, Artificial Sequence
  EGFdomain
atcaccatcagacacgtgatggtgatggtgatggccggcgggcc

SEQ ID NO: 27 - DNA, 42 bp, Artificial Sequence
  EGFdomain
tgatcaatgtgcttcttctcatgtcaaaacggtggttcttgt

SEQ ID NO: 28 - DNA, 43 bp, Artificial Sequence
  EGFdomain
ccttacaagaaccaccgttttgacatggagaagaagcacattg

SEQ ID NO: 29 - DNA, 43 bp, Artificial Sequence
  EGFdomain
aaggaccaattgcaatcttacatttgtttctgtttgccagctt

SEQ ID NO: 30 - DNA, 43 bp, Artificial Sequence
  EGFdomain
tcgaaagctggcaaacagaaacaaatgtaagattgcaattggt

SEQ ID NO: 31 - DNA, 42 bp, Artificial Sequence
  EGFdomain
tcgaaggtagaaactgtgaactcataaggatgactaaggtac

SEQ ID NO: 32 - DNA, 35 bp, Artificial Sequence
  EGFdomain
cttagtcatccttatgagtttcacagtttctacct

SEQ ID NO: 33 - DNA, 30 bp, Artificial Sequence
  primerM
accttcgaaacgatgtcgtcctctcgattc

SEQ ID NO: 34 - DNA, 28 bp, Artificial Sequence
  primerN
acctctagatcatttacccaatctattc

SEQ ID NO: 35 - DNA, 27 bp, Artificial Sequence
  primerO
tatggtaccatggcgtcagagaacaac

SEQ ID NO: 36 - DNA, 27 bp, Artificial Sequence
  primerP
cttgggcccttaagccttggcaacgtg

SEQ ID NO: 37 - DNA, 22 bp, Artificial Sequence
  primerQ
taagaattcatgggcgccgccg

SEQ ID NO: 38 - DNA, 26 bp, Artificial Sequence
  primerR
taagaattctcagaactcgtcccgca

SEQ ID NO: 39 - DNA, 27 bp, Artificial Sequence
  primerS
caactcgagatgtctgacaaatctgcc

SEQ ID NO: 40 - DNA, 27 bp, Artificial Sequence
  primerT
cttgggcccttaagccttggcaacgtg

SEQ ID NO: 41 - DNA, 27 bp, Artificial Sequence
  primerU
taatacgtacatcaccatcaccatcac

SEQ ID NO: 42 - DNA, 30 bp, Artificial Sequence
  primerV
taagaattcttagtcatccttatgagtttc